(19) United States
(12) Patent Application Publication
(10) Pub. No.: US 2022/0031236 A1
(43) Pub. Date: Feb. 3, 2022

(54) INTOXICATION IDENTIFICATION SYSTEMS AND METHODS

(71) Applicant: DENSO International America, Inc., Southfield, MI (US)

(72) Inventors: Sibu VARUGHESE, Sterling Heights, MI (US); Martin NESPOLO, Grosse Pointe Woods, MI (US); Gareth WEBB, New Hudson, MI (US); Thomas KRZYZAK, Livonia, MI (US); Wilson YIM, Troy, MI (US); Matthew JOHNSON, Royal Oak, MI (US)

(21) Appl. No.: 16/942,984

(22) Filed: Jul. 30, 2020

(51) Int. Cl. A61B 5/00; A61B 5/18; A61B 5/024

(52) U.S. Cl. CPC A61B 5/4845; A61B 5/18; A61B 5/0022; A61B 2562/0219; A61B 5/4266; A61B 5/024; A61B 5/749; A61B 5/0013

(57) ABSTRACT

An ordering module is configured to receive an order for delivery of one or more intoxicants to a user of a mobile device by an intoxicant vendor. An intoxication module is configured to determine an intoxication level of the user of the mobile device based on at least one of: perspiration of the user measured using a perspiration sensor of a first wearable device; a heart rate of the user measured using a heartrate sensor of a second wearable device; an acceleration of the mobile device measured using an accelerometer of the mobile device; a voice input captured using a microphone of the mobile device; and an image of the user captured using a camera of the mobile device. The ordering module is configured to transmit the order for delivery of the one or more intoxicants and the intoxication level of the user to the intoxicant vendor via a network.

### FIELD

The present disclosure relates to mobile devices, such as mobile devices configured to interact with vehicles.

### BACKGROUND

This section provides background information related to the present disclosure which is not necessarily prior art.

Passive entry/passive start (PEPS) systems allow a vehicle to locate a key associated with the vehicle, such as a wireless key fob carried by a user of the vehicle. PEPS systems can allow anyone in possession of a key fob that has been previously paired with a vehicle's central PEPS electronic control module (ECM) to access the vehicle by grabbing a door handle and to start the vehicle with a push of a button.

The central PEPS ECM authenticates the key fob to determine if the key fob is authorized to access the vehicle (e.g., previously paired with the central PEPs ECM). The central PEPS ECM uses a signal strength determined using a plurality of vehicle antennas to estimate a location of the key fob. If the key fob can be authenticated and is located within a predetermined area, the vehicle's function is made available to the user, such as the vehicle doors are unlocked and/or the vehicle is started.

### SUMMARY

This section provides a general summary of the disclosure, and is not a comprehensive disclosure of its full scope or all of its features.

In a feature, an intoxicant ordering system includes: an ordering module configured to receive an order for delivery of one or more intoxicants to a user of a mobile device by an intoxicant vendor; an intoxication module configured to determine an intoxication level of the user of the mobile device based on at least one of: perspiration of the user measured using a perspiration sensor of a first wearable device; a heart rate of the user measured using a heartrate sensor of a second wearable device; an acceleration of the mobile device measured using an accelerometer of the mobile device; a voice input captured using a microphone of the mobile device; and an image of the user captured using a camera of the mobile device, where the ordering module is configured to transmit the order for delivery of the one or more intoxicants and the intoxication level of the user to the intoxicant vendor via a network.

In further features, the intoxication module is configured to determine the intoxication level of the user based on the perspiration of the user measured using the perspiration sensor of a wearable device and reference perspiration data.

In further features, the intoxication module is configured to determine the intoxication level of the user based on the heart rate of the user and a reference heart rate.

In further features, the reference heart rate is a predetermined fixed heart rate.

In further features, a reference module is configured to selectively adjust the reference heart rate based on the heart rate of the user measured using the heartrate sensor of the wearable device.

In further features, the intoxication module is configured to increase the intoxication level of the user as the heart rate of the user becomes greater than the reference heart rate and to decrease the intoxication level of the user as the heart rate of the user decreases toward the reference heart rate.

In further features, the intoxication module is configured to determine the intoxication level of the user based on the acceleration of the mobile device and a reference acceleration.

In further features, the reference acceleration is a predetermined fixed acceleration.

In further features, a reference module is configured to selectively adjust the reference acceleration based on the acceleration of the mobile device measured using the accelerometer of the mobile device.

In further features, the intoxication module is configured to increase the intoxication level of the user as the acceleration becomes greater than the reference acceleration and to decrease the intoxication level of the user as the acceleration decreases toward the reference acceleration.

In further features, the intoxication module is configured to determine the intoxication level of the user based on the voice input and a reference voice input.

In further features, the reference voice input is a predetermined fixed voice input including one or more spoken words.

In further features, a reference module is configured to selectively adjust the reference voice input based on voice input captured using the microphone of the mobile device.

In further features, the intoxication module is configured to increase the intoxication level of the user as differences between the voice input and the reference voice input increase.

In further features, the intoxication module is configured to determine the intoxication level of the user based on the image of the user and a reference image of the user.

In further features, the reference image of the user is a predetermined fixed image of the user.

In further features, a reference module is configured to selectively adjust the reference image based on the image of the user.

In further features, a vehicle system includes: the intoxicant ordering system; and a vehicle configured to: wirelessly connect to the mobile device; and selectively at least one of: unlock one or more doors of the vehicle; and start the vehicle.

In further features, the intoxication module is configured to determine the intoxication level of the user of the mobile device based on at least two of: the perspiration of the user measured using the perspiration sensor of the first wearable device; the heart rate of the user measured using the heartrate sensor of the second wearable device; the acceleration of the mobile device measured using the accelerometer of the mobile device; the voice input captured using the microphone of the mobile device; and the image of the user captured using the camera of the mobile device.

In a feature, an intoxicant ordering method includes: by a mobile device, receiving an order for delivery of one or more intoxicants to a user of the mobile device by an intoxicant vendor; by the mobile device, determining an intoxication level of the user of the mobile device based on at least one of: perspiration of the user measured using a perspiration sensor of a first wearable device; a heart rate of the user measured using a heartrate sensor of a second wearable device; an acceleration of the mobile device measured using an accelerometer of the mobile device; a voice input captured using a microphone of the mobile device; and an image of the user captured using a camera of the mobile device, by the mobile device, transmitting the order for delivery of the one or more intoxicants and the intoxication level of the user to the intoxicant vendor via a network.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a functional block diagram of a subject vehicle with a localization system;

FIG. 2 is a functional block diagram including example components of a portable device;

FIG. 3 is a functional block diagram of an example intoxicant ordering system;

FIG. 4 is a functional block diagram of an example implementation of a portable device; and

FIG. 5 is a flowchart depicting an example method of ordering and transmitting an order for one or more intoxicants to a vendor of the intoxicant.

Corresponding reference numerals indicate corresponding parts throughout the several views of the drawings.

### DETAILED DESCRIPTION

Example embodiments will now be described more fully with reference to the accompanying drawings.

Mobile devices can be used to perform various functions, such as perform internet searches, check email, send messages, etc. Examples of mobile devices include, but are not limited to, such as (smart) cellular phones and tablet devices.

Mobile devices can also be used to perform other functions, such as to order food and delivery of one or more intoxicants. For example, via a mobile device, a user can place an order for delivery of one or more intoxicants (e.g., alcoholic beverages) by a vendor to the user. The user can order delivery of the intoxicant(s), for example, to a home of the user from a vendor located outside of the home or to a seat of the user within a stadium that the vendor is also located within.

The present application involves the mobile device determining an intoxication level of the user based on one or more measurements and transmitting the intoxication level of the user with the order for delivery of the intoxicant(s). Examples of the measurements include, but are not limited to, one or more of perspiration of the user measured using a wearable device, a heart rate of the user measured using a wearable device, voice input of the user captured using a microphone, an image of the user captured using a camera, and an acceleration of the mobile device.

The intoxication level of the user can help a deliverer (from the vendor) of the intoxicant(s) determine whether the user is too intoxicated for delivery of the intoxicant(s). One or more other actions may additionally or alternatively be taken based on the intoxication level of the user. For example, when the intoxication level of the user is high, a suggestion may be presented to the user for delivery of one or more less intoxicating substances (e.g., lower or zero alcohol beverages) and/or for use of a ride share service for travel.

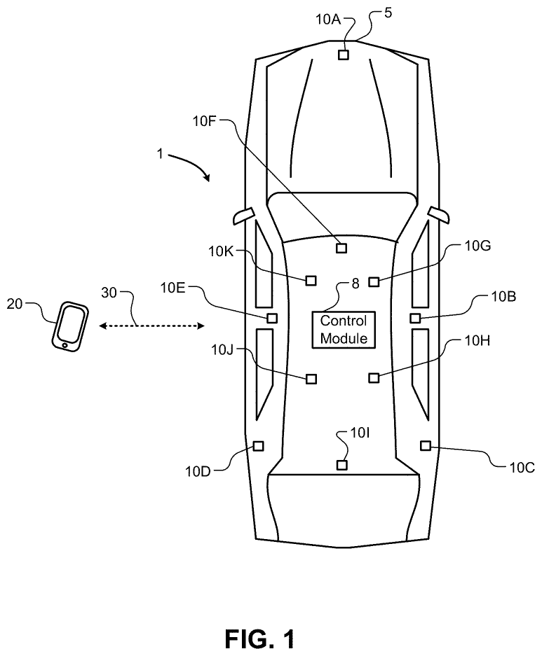

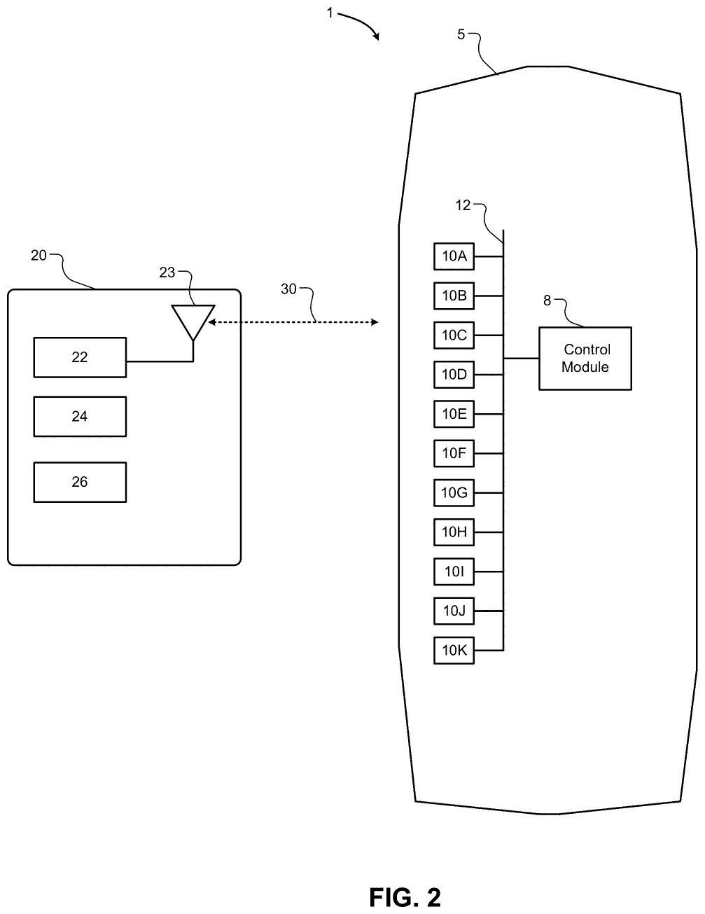

With reference to FIGS. 1 and 2, a localization system 1 is provided within a vehicle 5 and includes a control module 8 (also referred to as a central communication module) and a plurality of sensors 10A to 10K (collectively referred to as sensors 10). The sensors 10 can include a number of sensors that are mounted on or to the exterior of the vehicle 5, such as exterior body or trim components of the vehicle 5. In the example of FIG. 1, sensors 10A to 10E are shown as exterior sensors. The sensors 10 can additionally or alternatively include a number of sensors that are mounted on or to the interior of the vehicle 5, such as interior trim components of the vehicle 5. In the example of FIG. 1, sensors 10F to 10K are shown as interior sensors. For example only, the sensors 10 may include antennas or antenna modules.

The control module 8 can be implemented, for example, as a PEPS electronic control module (ECM) and is described in further detail below. The control module 8 can communicate with the sensors 10 using a wired vehicle interface 12, such as in the example of FIG. 2. The vehicle interface 12, for example, can include a controller area network (CAN) bus and/or a lower data rate communication bus, such as a local interconnect network (LIN) bus. The vehicle interface 12 can also include a clock extension peripheral interface (CXPI) bus. Additionally or alternatively, the vehicle interface 12 can include a combination of CAN bus, LIN bus, and CXPI bus communication interfaces. Additionally or alternatively, the control module 8 can communicate with the sensors 10 using wireless communication.

The control module 8 is configured to establish a secure communication connection, such as a Bluetooth low energy (BLE) communication connection, for example, with a portable device 20. As noted above, the control module 8 communicates information about the secure communication connection to the sensors 10 via the vehicle interface 12, such as the timing of the next communication connection event, the timing interval between communication connection events, the communication channel for the next communication connection event, a channel map, a channel hop interval or offset to calculate the channel for subsequent communication connection events, communication latency information, and/or communication jitter information, etc. The sensors 10 can then follow the secure communication connection between the control module 8 and the portable device 20 and receive communication packets sent by the portable device 20 to the control module 8. The sensors 10 can then measure signal information about the signals received from the portable device, such as relative signal strength (RSSI), angle of arrival, time of arrival, time difference of arrival, etc., and communicate the signal information to the control module 8 via communication over the vehicle interface 12.

With particular reference to FIG. 2, the portable device 20 can include a communication chipset 22, such as a BLE chipset, a WiFi chipset, or a WiFi direct chipset, connected to one or more antennas, such as antenna 23. The portable device 20 can also include application software stored in memory 24. The portable device 20 can also optionally include a global positioning system (GPS) module 26 or another suitable type of location device. The portable device 20 sends and receives communication signals 30 to and from the control module 8. As discussed above, the sensors 10 can listen for the communication signals 30 based on the information about the secure communication connection received from the control module 8. As such, the sensors 10 also receive the communication signals 30 sent by the portable device 20 to the control module 8. The portable device 20 can also be referred to as a mobile device.

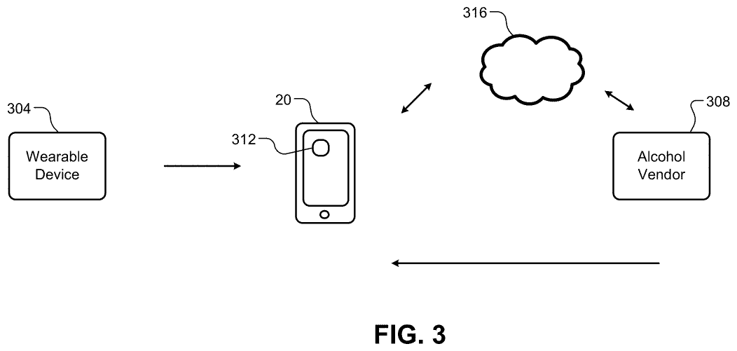

Examples of the portable device 20 include cellular (smart) phones, tablet devices, and other types of devices. In addition to being used to interact with the vehicle 10, the portable device 20 can also be used to perform one or more other operations/functions. For example, as shown in FIG. 3, the portable device 20 can wirelessly interact with one or more wearable devices, such as wearable device 304. Additionally, the portable device 20 can place orders with various different types of vendors. For example, the portable device 20 can be used to place food orders with food vendors via one or more food ordering applications.

As another example, the portable device 20 can be used to place orders for one or more alcoholic beverages with vendors of alcohol, such as alcohol vendor 308. Orders for alcoholic beverages may be placed via an application 312 executed by the portable device 20. The alcohol vendor 308 may deliver the ordered alcoholic beverage(s) to the location of the portable device 20 or another user specified location in response to the order. The alcohol vendor 308 may be located at a separate building from a user of the portable device 20. For example, the user may use the portable device 20 at home to order one or more alcoholic beverages to be delivered by a retailer (e.g., a grocer, convenience store, etc.) to the home of the user. As another example, the user may use the portable device 20 to order one or more alcoholic beverages to be delivered to the user at a seat of the user within a stadium by an employee of the stadium or an establishment within the stadium.

The portable device 20 may communicate with the alcohol vendor 308 via one or more networks 316, such as a local area network (LAN), a wide area network (WAN) (e.g., the Internet), or a combination.

Alcohol vendors, however, may be required to not supply alcoholic beverages (and not fulfill orders for alcoholic beverages) to users that are intoxicated. When an order for one or more alcoholic beverages is placed through the application, the alcohol vendor 308 does not know whether the user of the portable device 20 is intoxicated or not until the point of delivery of the ordered alcoholic beverages to the user.

There is a need to identify intoxication levels of users ordering alcoholic beverages via an application to protect alcohol vendors, the users, and the public generally.

The present application involves a portable device (e.g., a mobile phone) configured to determine an intoxication level of a user placing an order for one or more alcoholic beverages (or other intoxicant) via an application. The portable device is configured to determine the intoxication level based on at least one of: data collected via one or more wearable electronic devices; and data from one or more sensors of the portable device. Examples of data collected via one or more wearable electronic devices include heart rate and perspiration. Examples of data collected via the portable device include voice recordings captured by a microphone, images captured using a camera, and gyroscope/accelerometer measurements.

The portable device is configured to transmit the intoxication level of the user placing the order along with the order to an alcohol vendor. One or more actions can be taken based on the intoxication level of the user. For example, if the intoxication level of the user is greater than a predetermined value, the alcohol vendor can refuse (and not fulfil) the order or assess the user more closely for intoxication upon delivery of the order to the user. Additionally or alternatively, the alcohol vender can recommend one or more alternative beverages (e.g., beverages having lower or zero alcohol content) when the intoxication level is greater than a predetermined value. Additionally or alternatively, the portable device may output one or more recommendations to the user when the intoxication level of the user is greater than the predetermined value. Examples of recommendations include not driving and instead hiring a ride, such as via a ride sharing service (e.g., Uber, Lyft, etc.).

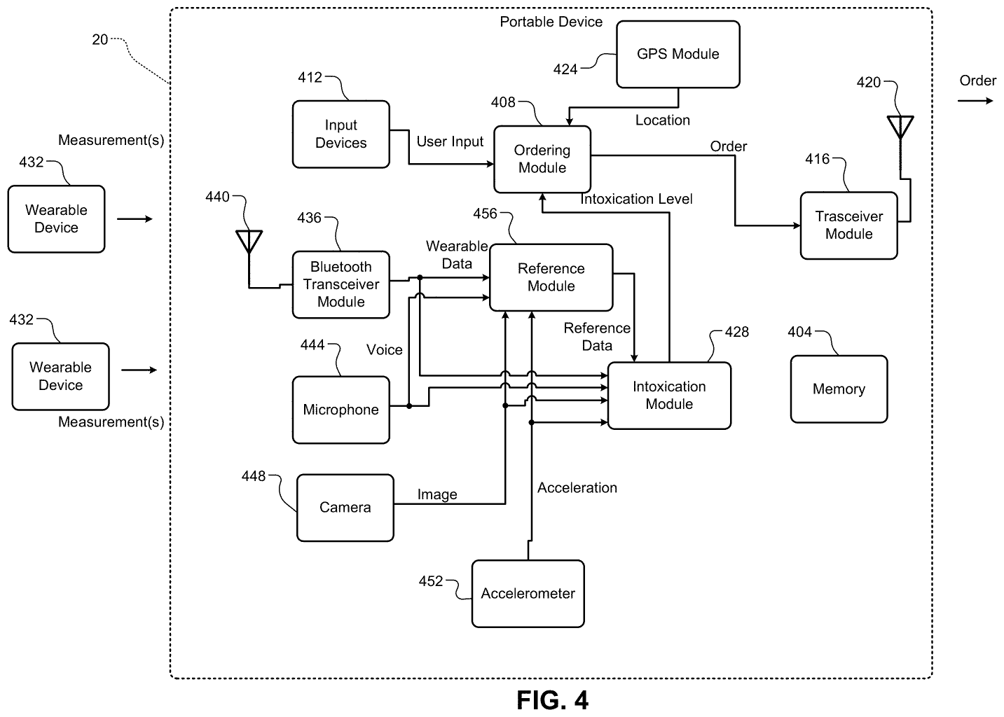

FIG. 4 is a functional block diagram of an example implementation of the portable device 20.

The portable device 20 includes memory 404 storing the application 312 for ordering alcoholic beverages via vendors, such as the alcohol vendor 308. An ordering module 408 executes the application to create and transmit an order for one or more alcoholic beverages to a vendor, such as the alcohol vendor 308. The ordering module 408 may allow the user to select which alcohol vendor to order from via the application 312 if multiple different vendors are available. In various implementations, the application 312 may be tailored to ordering from one alcohol vendor.

The ordering module 408 generates the order based on user input from one or more user input devices 412 of the portable device 20. Examples of the user input devices include, but are not limited to, buttons and switches that are actuatable by users of the portable device 20. In various implementations, the portable device 20 includes a touchscreen display, and the ordering module 408 may receive user input via touching of the touchscreen display.

The order module 408 transmits the order (including one or more alcoholic beverages) to the alcohol vendor 308. For example, the portable device 20 includes a transceiver 416 that transmits the order wirelessly to the alcohol vendor 308. The transceiver 416 may be, for example, a cellular transceiver, a WiFi transceiver, or another suitable type of transceiver. The transceiver 416 transmits the order via one or more antennas, such as antenna 420.

The ordering module 408 also includes a present intoxication level (value) of the user of the portable device 20 with or in the order. The ordering module 408 also includes in the order a location for delivery of the order to the user. The location may be, for example, a location stored in the memory 404 in association with the user or a present location of the portable device 20, such as provided by a GPS module 424 of the portable device 20. The GPS module 424 may determine the present location of the portable device 20 using signals from satellites, such as using triangulation. The ordering module 408 may also include payment information for the order in the order. Alternatively, payment for the order may be collected upon delivery to the user if the user is determined to be not intoxicated by a deliverer of the order. The payment information may be stored in the memory 404 or received via one or more of the input devices 412.

An intoxication module 428 determines the (present) intoxication level of the user of the portable device 20. In various implementations, the intoxication level may be expressed as a value within a range from 0 to 100, where 0 corresponds to no intoxication and 100 corresponds to a predetermined amount of intoxication (e.g., a predetermined blood alcohol content).

The intoxication module 428 determines the intoxication level of the user based on at least one of: wearable data from one or more wearable electronic devices 432; and data from one or more sensors of the portable device 20. The intoxication module 428 determines the intoxication level of the user based on comparisons of the received data with respective reference data.

Examples of the wearable data include an indicator of whether the user is perspiring or not (or a perspiration rate of the user), a heart rate of the user, and other types of parameters measured by sensors of wearable electronic devices. Examples of the wearable electronic devices 432 include smart watches, fitness trackers, heart rate trackers, perspiration sensors, and other types of wearable electronic devices. The wearable electronic devices 432 are configured to be worn on bodies of users.

A Bluetooth transceiver 436 may wirelessly receive the wearable data from the wearable electronic devices 432 via one or more antennas, such as antenna 440. While the example of receipt of the wearable data via a Bluetooth connection is provided, the wearable data may additionally or alternatively be received wirelessly in another type of wireless connection, such as via a WiFi (IEEE 802.11 based) connection, a ZigBee (IEEE 802.15.4 based) connection, etc.

Examples of data from one or more sensors of the portable device 20 include voice input captured using a microphone 444 of the portable device 20, an image of the user captured using a camera 448 of the portable device 20, and acceleration (e.g., x, y, an z axis acceleration) measured by an accelerometer 452 of the portable device. In addition to or as alternative to the accelerometer 452, the portable device 20 may include a gyroscope. The voice input may be a predetermined string of one or more words prompted to be spoken by the ordering module 408 or a non-specified words spoken by the user. The image may be, for example, taken of a face of the user. The accelerometer 452 is configured to measure acceleration of the portable device 20 in one or more directions, such as in x, y, and z directions.

A reference module 456 provides the reference data. The intoxication module 428 may determine differences between the data received (wearable data and/or data from sensors of the portable device 20) with respective reference data from the reference module 456.

For example, the intoxication module 428 may determine a difference between a heart rate of the user received from a wearable device and a reference heart rate of the user if the heart rate of the user is received. The intoxication module 428 may determine a difference between a perspiration amount of the user and a reference perspiration amount of the user if a perspiration amount of the user is received. The intoxication module 428 may determine a difference between a voice input from the user and a reference voice input from the user if the voice input is received. The intoxication module 428 may determine a difference between the image of the user and a reference image of the user if the image is received. The intoxication module 428 may determine a difference between the acceleration of the portable device 20 and a reference acceleration of the portable device 20 if an acceleration is received.

The reference data may represent the user not being intoxicated (e.g., intoxication value=0). The reference data may be predetermined data and may be fixed. In this example, the reference data may be generated based on a group of people, such as the general public.

In various implementations, the reference module 456 may selectively adjust the reference data based on data received from the sensors of the portable device 20 or the wearable data. This may adapt the reference data to be specific to the user of the portable device 20.

For example, the reference module 456 may selectively adjust the reference perspiration data based on perspiration data received from a wearable device while the user is not intoxicated. The reference module 456 may selectively adjust the reference heart rate data based on heart rate data received from a wearable device while the user is not intoxicated. The reference module 456 may selectively adjust the reference voice data based on voice data received captured via the microphone 444 while the user is not intoxicated. The reference module 456 may selectively adjust the reference image data based on one or more images captured via the camera 448 while the user is not intoxicated. The reference module 456 may selectively adjust the reference acceleration data based on acceleration data received captured via the accelerometer 452 while the user is not intoxicated.

The intoxication module 428 may determine the intoxication level of the user using, for example, one or more equations and/or lookup tables that relate the available ones of the differences to intoxication levels. For example, the intoxication module 428 may increase the intoxication level as the heart rate of the user increases above the reference heart rate and vice versa. Additionally or alternatively, the intoxication module 428 may increase the intoxication level as the difference between the perspiration and the reference perspiration increases and vice versa. Additionally or alternatively, the intoxication module 428 may increase the intoxication level as the difference between voice data and the reference voice data increases and vice versa. Additionally or alternatively, the intoxication module 428 may increase the intoxication level as the difference between the acceleration and the reference acceleration increases and vice versa. Additionally or alternatively, the intoxication module 428 may increase the intoxication level as the difference between the image and the reference image increases and vice versa.

As discussed above, the ordering module 408 includes the intoxication level of the user with the order transmitted to the alcohol vendor 308. One or more actions may be taken based on the intoxication level of the user. For example, when the intoxication level is greater than a predetermined value (e.g., 50 in the example of intoxication values being between 0 and 100), the ordering module 408 may output one or more recommendations (e.g., beverages with less or no alcohol) to the user. Additionally or alternatively, the ordering module 408 may output a recommendation to use a vehicle sharing application and not driving when the intoxication level is greater than the predetermined value. As used herein, the output may include visually displaying information on a display, audibly outputting information via one or more speakers, or a combination of audible and visual output. The intoxication level of the user of the portable device 20 may aid the alcohol vendor in determining whether to transfer the ordered beverage(s) to the user at the point of deliver.

The ordering module 408 may transmit the intoxication level to the alcohol vendor 308. In various implementations, the ordering module 408 may transmit an indicator of a general range of the intoxication level to the alcohol vendor 308. For example, the identifier may be a color indicative of a range of the intoxication level of the user. For example, the intoxication module 428 may set the indicator to a first color (e.g., green) when the intoxication level is less than a first predetermined value (e.g., 25) and to a second color (e.g., red) when the intoxication level is greater than a second predetermined value (e.g., 50) that is greater than the first predetermined value. The intoxication module 428 may set the indicator to a third color (e.g., yellow) when the intoxication level is between the first and second predetermined values. While the example of using colors as the indicator is provided, another suitable indicator may be used. Also, while the example of alcohol is used herein, the present application is also applicable to orders for delivery of other types of intoxicants, such as marijuana, etc. Intoxicants include substances that cause humans to become intoxicated.

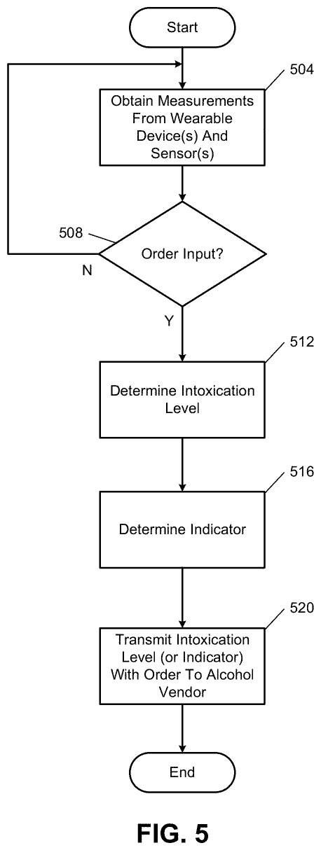

FIG. 5 is a flowchart depicting an example method of generating and transmitting an order for one or more alcoholic beverages to an alcohol vendor. Control begins with 504 where the portable device 20 obtains the measurements from one or more sensors of the portable device 20 (e.g., voice input, an image of the user, acceleration data, etc.) and/or measurements from one or more wearable devices, such as perspiration and/or heartrate.

At 508, the ordering module 408 determines whether the user has completed an order for one or more intoxicants (e.g., beverages including alcohol) for delivery to a user of the portable device 20. The user may complete an order for the one or more intoxicants via one or more of the input devices 412. If 508 is false, control may return to 504. If 508 is true, the user has input an order for one or more intoxicants for delivery to the user by a vendor of the intoxicant(s), and control may continue with 512.

At 512, the intoxication module 428 determines the intoxication level of the user based on the measurement(s) and the respective reference data, as discussed above. At 516, the intoxication module 428 may determine the indicator of the general range of intoxication of the user based on the intoxication level of the user. For example, the intoxication module 428 may set the indicator to the first color (e.g., green) when the intoxication level is less than the first predetermined value, set the indicator to the second color (e.g., red) when the intoxication level is greater than the second predetermined value, and set the indicator to the third color (e.g., yellow) when the intoxication level is between the first and second predetermined values.

At 520, the ordering module 408 transmits the order for the one or more intoxicants and the intoxication level, the indicator, or both to the vendor of the intoxicant. The vendor of the intoxicant may be located within a different building than the user (e.g., in the example of home delivery by a separate business) or within the same building as the user (e.g., in the example of ordering within a stadium).

In a feature, an intoxicant ordering system includes: an ordering module configured to receive an order for delivery of one or more intoxicants to a user of a mobile device by an intoxicant vendor; an intoxication module configured to determine an intoxication level of the user of the mobile device based on at least one of: perspiration of the user measured using a perspiration sensor of a first wearable device; a heart rate of the user measured using a heartrate sensor of a second wearable device; an acceleration of the mobile device measured using an accelerometer of the mobile device; a voice input captured using a microphone of the mobile device; and an image of the user captured using a camera of the mobile device, where the ordering module is configured to transmit the order for delivery of the one or more intoxicants and the intoxication level of the user to the intoxicant vendor via a network.

In further features, the intoxication module is configured to determine the intoxication level of the user based on the perspiration of the user measured using the perspiration sensor of a wearable device and reference perspiration data.

In further features, the intoxication module is configured to determine the intoxication level of the user based on the heart rate of the user and a reference heart rate.

In further features, the reference heart rate is a predetermined fixed heart rate.

In further features, a reference module is configured to selectively adjust the reference heart rate based on the heart rate of the user measured using the heartrate sensor of the wearable device.

In further features, the intoxication module is configured to increase the intoxication level of the user as the heart rate of the user becomes greater than the reference heart rate and to decrease the intoxication level of the user as the heart rate of the user decreases toward the reference heart rate.

In further features, the intoxication module is configured to determine the intoxication level of the user based on the acceleration of the mobile device and a reference acceleration.

In further features, the reference acceleration is a predetermined fixed acceleration.

In further features, a reference module is configured to selectively adjust the reference acceleration based on the acceleration of the mobile device measured using the accelerometer of the mobile device.

In further features, the intoxication module is configured to increase the intoxication level of the user as the acceleration becomes greater than the reference acceleration and to decrease the intoxication level of the user as the acceleration decreases toward the reference acceleration.

In further features, the intoxication module is configured to determine the intoxication level of the user based on the voice input and a reference voice input.

In further features, the reference voice input is a predetermined fixed voice input including one or more spoken words.

In further features, a reference module is configured to selectively adjust the reference voice input based on voice input captured using the microphone of the mobile device.

In further features, the intoxication module is configured to increase the intoxication level of the user as differences between the voice input and the reference voice input increase.

In further features, the intoxication module is configured to determine the intoxication level of the user based on the image of the user and a reference image of the user.

In further features, the reference image of the user is a predetermined fixed image of the user.

In further features, a reference module is configured to selectively adjust the reference image based on the image of the user.

In further features, a vehicle system includes: the intoxicant ordering system; and a vehicle configured to: wirelessly connect to the mobile device; and selectively at least one of: unlock one or more doors of the vehicle; and start the vehicle.

In further features, the intoxication module is configured to determine the intoxication level of the user of the mobile device based on at least two of: the perspiration of the user measured using the perspiration sensor of the first wearable device; the heart rate of the user measured using the heartrate sensor of the second wearable device; the acceleration of the mobile device measured using the accelerometer of the mobile device; the voice input captured using the microphone of the mobile device; and the image of the user captured using the camera of the mobile device.

In a feature, an intoxicant ordering method includes: by a mobile device, receiving an order for delivery of one or more intoxicants to a user of the mobile device by an intoxicant vendor; by the mobile device, determining an intoxication level of the user of the mobile device based on at least one of: perspiration of the user measured using a perspiration sensor of a first wearable device; a heart rate of the user measured using a heartrate sensor of a second wearable device; an acceleration of the mobile device measured using an accelerometer of the mobile device; a voice input captured using a microphone of the mobile device; and an image of the user captured using a camera of the mobile device, by the mobile device, transmitting the order for delivery of the one or more intoxicants and the intoxication level of the user to the intoxicant vendor via a network.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

In this application, including the definitions below, the terms “module” and “system” may refer to, be part of, or include circuits or circuitry that may include processor hardware (shared, dedicated, or group) that executes code and memory hardware (shared, dedicated, or group) that stores code executed by the processor hardware. The code is configured to provide the features of the modules and systems described herein. In addition, in this application the terms “module” and “system” may be replaced with the term “circuit.” The term “memory hardware” may be a subset of the term computer-readable medium. The term computer-readable medium does not encompass transitory electrical and electromagnetic signals propagating through a medium, and may therefore be considered tangible and non-transitory. Non-limiting examples of a non-transitory tangible computer readable medium include nonvolatile memory, volatile memory, magnetic storage, and optical storage.

The apparatuses and methods described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as JavaScript Object Notation (JSON), hypertext markup language (HTML) or extensible markup language (XML); (ii) assembly code; (iii) object code generated from source code by a compiler; (iv) source code for execution by an interpreter; (v) source code for compilation and execution by a just-in-time compiler, etc. As examples only, source code may be written using syntax from languages including C, C++, C#, Objective C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

1. An intoxicant ordering system, comprising:
an ordering module configured to receive an order for delivery of one or more intoxicants to a user of a mobile device by an intoxicant vendor;
an intoxication module configured to determine an intoxication level of the user of the mobile device based on at least one of:
perspiration of the user measured using a perspiration sensor of a first wearable device;
a heart rate of the user measured using a heartrate sensor of a second wearable device;
an acceleration of the mobile device measured using an accelerometer of the mobile device;
a voice input captured using a microphone of the mobile device; and
an image of the user captured using a camera of the mobile device,
wherein the ordering module is configured to transmit the order for delivery of the one or more intoxicants and the intoxication level of the user to the intoxicant vendor via a network.

2. The intoxicant ordering system of claim 1 wherein the intoxication module is configured to determine the intoxication level of the user based on the perspiration of the user measured using the perspiration sensor of a wearable device and reference perspiration data.

3. The intoxicant ordering system of claim 1 wherein the intoxication module is configured to determine the intoxication level of the user based on the heart rate of the user and a reference heart rate.

4. The intoxicant ordering system of claim 3 wherein the reference heart rate is a predetermined fixed heart rate.

5. The intoxicant ordering system of claim 3 further comprising a reference module configured to selectively adjust the reference heart rate based on the heart rate of the user measured using the heartrate sensor of the wearable device.

6. The intoxicant ordering system of claim 3 wherein the intoxication module is configured to increase the intoxication level of the user as the heart rate of the user becomes greater than the reference heart rate and to decrease the intoxication level of the user as the heart rate of the user decreases toward the reference heart rate.

7. The intoxicant ordering system of claim 1 wherein the intoxication module is configured to determine the intoxication level of the user based on the acceleration of the mobile device and a reference acceleration.

8. The intoxicant ordering system of claim 7 wherein the reference acceleration is a predetermined fixed acceleration.

9. The intoxicant ordering system of claim 7 further comprising a reference module configured to selectively adjust the reference acceleration based on the acceleration of the mobile device measured using the accelerometer of the mobile device.

10. The intoxicant ordering system of claim 7 wherein the intoxication module is configured to increase the intoxication level of the user as the acceleration becomes greater than the reference acceleration and to decrease the intoxication level of the user as the acceleration decreases toward the reference acceleration.

11. The intoxicant ordering system of claim 1 wherein the intoxication module is configured to determine the intoxication level of the user based on the voice input and a reference voice input.

12. The intoxicant ordering system of claim 11 wherein the reference voice input is a predetermined fixed voice input including one or more spoken words.

13. The intoxicant ordering system of claim 11 further comprising a reference module configured to selectively adjust the reference voice input based on voice input captured using the microphone of the mobile device.

14. The intoxicant ordering system of claim 11 wherein the intoxication module is configured to increase the intoxication level of the user as differences between the voice input and the reference voice input increase.

15. The intoxicant ordering system of claim 1 wherein the intoxication module is configured to determine the intoxication level of the user based on the image of the user and a reference image of the user.

16. The intoxicant ordering system of claim 15 wherein the reference image of the user is a predetermined fixed image of the user.

17. The intoxicant ordering system of claim 15 further comprising a reference module configured to selectively adjust the reference image based on the image of the user.

18. A vehicle system comprising:
the intoxicant ordering system of claim 1; and
a vehicle configured to:
wirelessly connect to the mobile device; and
selectively at least one of:
unlock one or more doors of the vehicle; and
start the vehicle.

19. The intoxicant ordering system of claim 1 wherein the intoxication module is configured to determine the intoxication level of the user of the mobile device based on at least two of:
the perspiration of the user measured using the perspiration sensor of the first wearable device;
the heart rate of the user measured using the heartrate sensor of the second wearable device;
the acceleration of the mobile device measured using the accelerometer of the mobile device;
the voice input captured using the microphone of the mobile device; and
the image of the user captured using the camera of the mobile device.

20. An intoxicant ordering method, comprising:
by a mobile device, receiving an order for delivery of one or more intoxicants to a user of the mobile device by an intoxicant vendor;
by the mobile device, determining an intoxication level of the user of the mobile device based on at least one of:
perspiration of the user measured using a perspiration sensor of a first wearable device;
a heart rate of the user measured using a heartrate sensor of a second wearable device;
an acceleration of the mobile device measured using an accelerometer of the mobile device;
a voice input captured using a microphone of the mobile device; and
an image of the user captured using a camera of the mobile device,
by the mobile device, transmitting the order for delivery of the one or more intoxicants and the intoxication level of the user to the intoxicant vendor via a network.
